(19) United States
(12) Patent Application Publication
(10) Pub. No.: US 2007/0162049 A1
(43) Pub. Date: Jul. 12, 2007

(54) Baby shoulder vacuum cup

(76) Inventors: Kun Yuan Tong, Albany, GA (US); John Tsunglin Tong, Sacramento, CA (US)

Correspondence Address: Kun Yuan Tong, 2308 W. Doublegate Dr., Albany, GA, 31721, US

(21) Appl. No.: 11/327,201

(22) Filed: Jan. 6, 2006

(51) Int. Cl. A61B 17/42

(52) U.S. Cl. 606/123

(57) ABSTRACT

Shoulder dystocia is an obstetric emergency. It is unpredictable and can occur with a large baby or a small baby. There are several maneuvers that can be applied to help prevent injury to the brachial plexus. But injury to the brachial plexus can still occur. A baby shoulder vacuum cup with a vacuum tube has a non-circular-shaped, preferably elongated or oval-shaped, bottom opening to fit onto the baby's shoulder to pull down or rotate the baby's shoulder instead of forcibly pulling on the baby's head to prevent injury to the brachial plexus. Various types of handles can be attached to the baby shoulder vacuum cup.

### THE BACKGROUND OF THE INVENTION

Shoulder dystocia is an obstetric emergency. It is unpredictable. It can occur with a large baby or a small baby. Even though there are several maneuvers, such as the McRobert's maneuver and Wood's corkscrew maneuver to deal with the problem, there are still a few babies who suffer from transient or permanent brachial plexus injury.

### BRIEF DESCRIPTION OF THE INVENTION

The obstetric vacuum cup is available and designed for suction of the fetal head to assist in the delivery of the baby's head during childbirth. The obstetric vacuum cup is circular in shape and is not suitable for the suction of the baby's shoulder. The baby shoulder vacuum cup invention presented here has a non-circular-shaped, preferably elongated or oval-shaped, bottom opening to fit a baby's shoulder. It can be applied onto the baby's shoulder to assist in the delivery of the baby's shoulder to prevent injury to the brachial plexus in case of shoulder dystocia. Various types of handles can be attached to the baby shoulder vacuum cup.

### THE DETAILED DESCRIPTION OF DRAWINGS

FIG. 1 is a lateral perspective view of a baby shoulder vacuum cup with a vacuum tube.

FIG. 2 is a bottom perspective view of a baby shoulder vacuum cup with a vacuum tube.

FIG. 3 is an oblique perspective view of a baby shoulder vacuum cup with a vacuum tube.

FIG. 4 is a perspective view of a baby shoulder vacuum cup that is placed onto a baby's shoulder and connected to a suction machine.

### THE DETAILED DESCRIPTION OF THE INVENTION

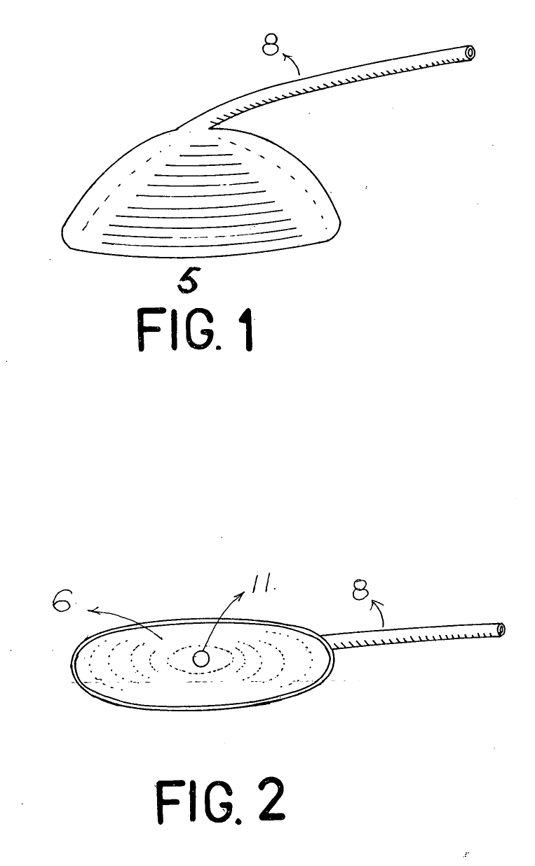

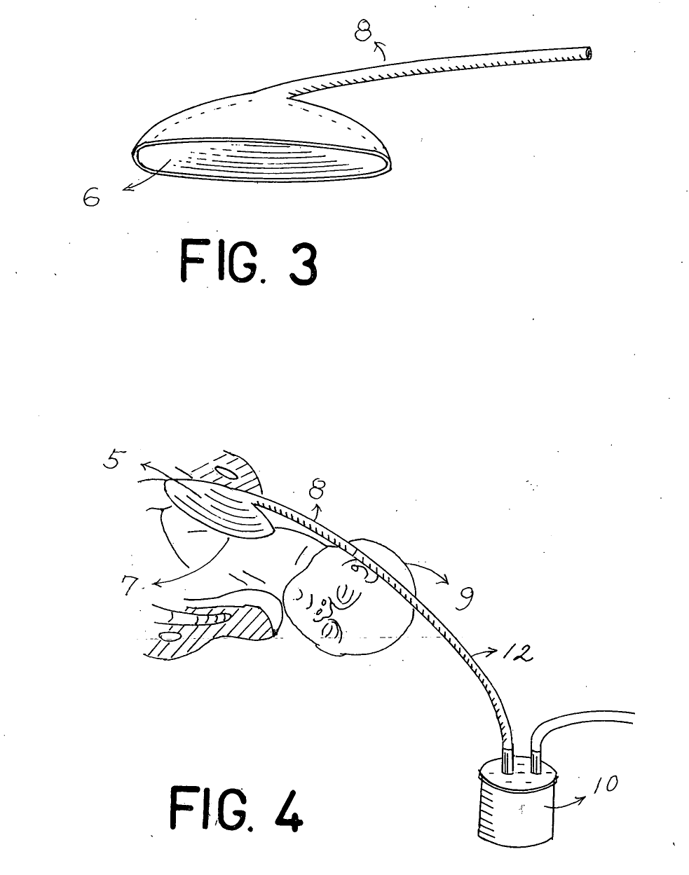

Referring now in detail to the drawings, numeral 5 of FIG. 1 is a baby shoulder vacuum cup with a vacuum tube 8. Numeral 6 of FIG. 2 is an oval-shaped bottom opening of baby shoulder vacuum cup 5 with a vacuum tube 8 to fit the baby's shoulder 7 of FIG. 4. Numeral 8 of FIGS. 1, 2, 3, and 4 is a vacuum tube of baby shoulder vacuum cup 5 which can be pulled on as a handle to facilitate traction and rotation of the baby's shoulder 7. Other handle types may be attached to the baby shoulder vacuum cup 5 to help facilitate pulling on the baby shoulder vacuum cup 5 (not drawn). In case of shoulder dystocia during a difficult delivery, an obstetrician can place a baby shoulder vacuum cup 5 with a vacuum tube 8 of FIGS. 1, 2, 3 and 4 onto the baby's shoulder 7 of FIG. 4 to pull down or rotate the baby's anterior shoulder 7, instead of forcibly pulling on the baby's head 9 of FIG. 4 to prevent injury to the brachial plexus. The vacuum tube 8 of FIGS. 1, 2, 3 and 4 is connected to a suction tube 12 of FIG. 4 that is connected to a suction machine 10 to create vacuum in the baby shoulder vacuum cup 5 as illustrated in FIG. 4. Numeral 11 of FIG. 2 is the opening of the vacuum tube 8 to the baby shoulder vacuum cup 5.

1. A baby shoulder vacuum cup comprising a vacuum cup with a non-circular bottom opening and a vacuum tube whereby said vacuum cup is applied to a baby's shoulder to facilitate traction and rotation of the baby's shoulder.

2. The baby shoulder vacuum cup of claim 1 wherein said vacuum cup has an oval-shaped bottom opening to fit onto the baby's shoulder.

3. The baby shoulder vacuum cup of claim 1 wherein said vacuum cup is of a variety of sizes to fit a variety of said baby's shoulder sizes.

4. The baby shoulder vacuum cup of claim 1 wherein said vacuum cup is of a variety of shapes to fit a variety of said baby's shoulder shapes.

5. The baby shoulder vacuum cup of claim 1 wherein said vacuum tube is to be connected to a suction device to create vacuum in said vacuum cup.

6. The baby shoulder vacuum cup of claim 1 wherein said vacuum cup is composed of polymeric material.

7. The baby shoulder vacuum cup of claim 1 further including a handle attached to said vacuum cup.

8. A baby shoulder vacuum cup comprising a vacuum cup with an elongated bottom opening and a vacuum tube whereby said vacuum cup is applied to a baby's shoulder to facilitate traction and rotation of the baby's shoulder.

9. The baby shoulder vacuum cup of claim 8 wherein said vacuum cup has an oval-shaped bottom opening to fit onto the baby's shoulder.

10. The baby shoulder vacuum cup of claim 8 wherein said vacuum cup is of a variety of sizes to fit a variety of said baby's shoulder sizes.

11. The baby shoulder vacuum cup of claim 8 wherein said vacuum cup is of a variety of shapes to fit a variety of said baby's shoulder shapes.

12. The baby shoulder vacuum cup of claim 8 wherein said vacuum tube is to be connected to a suction device to create vacuum in said vacuum cup.

13. The baby shoulder vacuum cup of claim 8 wherein said vacuum cup is composed of polymeric material.

14. The baby shoulder vacuum cup of claim 8 further including a handle attached to said vacuum cup.

15. A method of gripping a baby's shoulder comprising placing a baby shoulder vacuum cup with a non-circular bottom opening and a vacuum tube onto the baby's shoulder, connecting said vacuum tube to a suction machine, applying suction to said vacuum cup, grasping the baby's shoulder with said vacuum cup, whereby traction and rotation can be applied to the baby's shoulder.

16. The baby shoulder vacuum cup of claim 15 wherein said vacuum cup has an oval-shaped bottom opening to fit onto the baby's shoulder.

17. The baby shoulder vacuum cup of claim 15 wherein said vacuum cup is of a variety of sizes to fit a variety of said baby's shoulder sizes.

18. The baby shoulder vacuum cup of claim 15 wherein said vacuum cup is of a variety of shapes to fit a variety of said baby's shoulder shapes.

19. The baby shoulder vacuum cup of claim 15 wherein said vacuum tube is to be connected to a suction device to create vacuum in said vacuum cup.

20. The baby shoulder vacuum cup of claim 15 further including a handle attached to said vacuum cup.
